SEQUENCE LISTING (4 sequences)

SEQ ID NO: 1 - protein, 5 aa, Artificial Sequence
  GXXXG MOTIF
GXXXG

SEQ ID NO: 2 - protein, 32 aa, Homo sapiens
DVGSNKGAIIGLMVGGVVIATVIVITLVMLKK

SEQ ID NO: 3 - protein, 14 aa, Homo sapiens
GAIIGLMVGGVVIA

SEQ ID NO: 4 - protein, 26 aa, Homo sapiens
NKGAIIGLMVGGVVIATVIVITLVML

(19) United States
(12) Patent Application Publication
(10) Pub. No.: US 2010/0221759 A1
(43) Pub. Date: Sep. 2, 2010

(54) ASSAY FOR THE DIAGNOSIS OF ALZHEIMER'S DISEASE BASED ON THE DETERMINATION OF THE RATIO OF GAMMA-SECRETASE ABETA CLEAVAGE PRODUCTS

(75) Inventors: Gerd Multhaup, Kleinmachnow (DE); Lisa-Marie Münter, Berlin (DE)

Correspondence Address: PROSKAUER ROSE LLP, ONE INTERNATIONAL PLACE, BOSTON, MA, 02110, US

(73) Assignee: FU Berlin, Berlin (DE)

(21) Appl. No.: 12/303,898

(22) PCT Filed: Apr. 20, 2007
(86) PCT No.: PCT/EP07/03505

§ 371 Date: Dec. 8, 2008

(30) Foreign Application Priority Data
Jun. 8, 2006 (EP) 06 011 829.6

(51) Int. Cl. G01N 33/577; G01N 33/566

(52) U.S. Cl. 435/7.94; 436/501

(57) ABSTRACT

Described is a method for the diagnosis of Alzheimer's disease (AD) or a particular stage of the disease which is based on the determination of the ratio of at least two gamma-secretase cleavage products, Abeta48, Abeta45, Abeta42, Abeta38 and Abeta35, preferably the ratio of Abeta38: Abeta42. A decreased ratio of Abeta38: Abeta42 as compared to the normal ratio is indicative of AD. Moreover, kits suitable for carrying out said diagnostic method are described.

The present invention relates to a method for the diagnosis of Alzheimer's disease (AD) or a particular stage of the disease which is based on the determination of the ratio of at least two γ-secretase cleavage products, Abeta48, Abeta45, Abeta42, Abeta38 and Abeta35, preferably the ratio of Abeta38:Abeta42. A decreased ratio of Abeta38:beta42 as compared to the normal ratio is indicative of AD. The present invention also provides kits suitable for carrying out said diagnostic method.

For the diagnosis of Alzheimer's disease (AD) and for distinguishing AD from other dementia various methods are used. These diagnostic methods comprise the “scanning” of the brain using computer tomography (CT), magnetic resonance imaging (MRI) or positron emission tomography (PET) in order to visualize abnormalities in the brain. Another diagnostic approach is the mini-mental state examination (MMSE) which is based on the testing of particular cognitive problems (short-term memory, long-term memory, repetition tests), thus, allowing a distinct evaluation of the patient's capability as regards resolving of problems, linguistic competence etc. According to neurologists, geriatricians and psychiatrists who are familiar with a variety of dementia this test allows a precise diagnosis. Thus, MMSE forms the basis of current diagnosis.

An additional diagnostic method is based on the determination of compounds which are characteristic of the disease (biochemical markers, biomarkers) in body fluids like blood, liquor, urine etc. using selective laboratory tests. This approach allows the determination of changes of the concentrations due to the disease—even early in the course of the disease, when clinical symptoms might be mild and vague. However, this assay is problematic, since low concentrations of biochemical markers have to be determined selectively for early diagnosis and for exactly distinguishing between AD and other dementia. In order to enhance diagnostic accuracy, particular biochemical markers like the beta-amyloid protein and the tau protein are used. The cerebrospinal fluid (CSF) level of tau has been suggested to reflect neuronal and axonal degeneration or possibly formation of neurofibrillary tangles. Accordingly, tau is regarded as a general biomarker for a neuronal dysfunction (Creutzfeldt-Jacob, AD etc.). Tau levels moderately increase in AD (there is a stronger increase in CJD) and in other neurodegenerative diseases whereas phosphor-tau strongly increases in AD.

Currently, amyloid beta (Abeta) is regarded as the most important diagnostic parameter, since this peptide is responsible for the plaque like extra cellular depositions (amyloidosis) in the brain which is a characteristic feature early in the course of AD. APP is a type 1 transmembrane (TM) protein, which undergoes proteolytic processing by several secretases. First, the bulk of the ectodomain needs to be removed by membrane-bound alpha- or beta-secretases leading to secreted forms of APP and membrane bound C-terminal fragments alpha-CTF or beta-CTF, respectively. Regulated intramembrane proteolysis (RIP) of the beta-CTF by gamma-secretase occurs only after ectodomain shedding and releases the Abeta peptide from the membrane. The active gamma-secretase complex consists of the four subunits presenilin-1 (PS-1), APH-1, PEN-2 and nicastrin. PS-1 contains two catalytic active aspartate residues in TMS-6 and TMS-7. PS-1 is endoproteolytically cleaved and the N-terminal and C-terminal fragments were shown to exist as dimers in the catalytic core of gamma-secretase. The amino termini of CTF stubs are recognized by nicastrin, which functions as a gamma-secretase substrate receptor. The gamma-secretase cleaves at variable sites thus generating Abeta peptides of varying lengths. Abeta peptides are closely linked to AD as they accumulate to amyloid plaques. Abeta42 is the pathologically most relevant form of the Abeta species since it is more prone to aggregation and found elevated in AD patient brains.

A recently discussed mechanism of gamma-secretase cleavage implies sequential proteolytic cleavage steps to release Abeta. Accordingly, the first cut occurs at the cytoplasmic edge of the TMS at the epsilon-site, i.e. residue 49 of beta-CTF. The product Abeta49 remains membrane-bound and is further processed in a sequential action mode into Abeta46 (zeta-site) and Abeta40 or Abeta42 (gamma-site) (FIG. 1A). Notch was recently reported to be processed by gamma-secretase by a similar sequential mechanism. Previously it was shown that APP forms homodimers as it has been observed for other gamma-secretase substrates, e.g. the receptor tyrosine-protein kinase ErbB-4 and E-cadherin. The APP homodimerization is likely to be mediated by two different sites of the ectodomain, the loop region encompassing residues 91-111 and a second site overlapping with the collagen binding site spanning residues 448-465.

In a recent study, the levels of Abeta40 and Abeta42 of AD patients and of healthy individuals were compared. It could be shown that there were no differences in both groups as regards the levels of Abeta40, however AD patients showed decreased levels of Abeta42. Unfortunately, the absolute concentrations of Abeta42 observed in various AD patients differ dramatically (“high” individuals vs. “low” individuals). These variations of values of absolute concentrations show that the data obtained have been affected by various factors (state of the sample, assay system used, etc.). Accordingly, the establishing of limit values which is required for the development of a reliable diagnostic assay is almost impossible. Moreover, in the course of sporadic AD disease various levels of amyloid could be observed. Accordingly, the determination of the concentration of Abeta42 only is not sufficient for precise diagnosis of AD. In other words, at present, there is no reliable marker available for diagnosing AD, a particular stage of AD or for diagnosing an elevated risk for developing AD.

Thus, the technical problem underlying the present invention is to provide a marker for the reliable (early) diagnosis of Alzheimer's disease (AD) or a particular stage of said disease.

The solution to said technical problem is achieved by providing the embodiments characterized in the claims. During the experiments leading to the present invention a GxxxG motif could be identified mediating helix-helix interactions within the transmembrane segment of APP. This motif represents a unique homophilic interaction site of the gamma-secretase substrate CTF and controls the processing into Abeta via the final gamma-secretase cleavages. Mutations of this motif and subsequent analysis of Abeta species revealed that consecutive cuts occur by the two sets of cleavages identified, (I) 49-46-43-40-37-34 and (II) 48-45-42-38-35 (Abeta numbering). There is increasing evidence that these species are generated by consecutive helix-turn by helix-turn cleavages by the gamma-secretase starting from the C-terminus and progressing to the N-terminus of the substrate of beta-CTF. These findings have a direct impact on diagnostic approaches that monitor the progress of the disease. Accordingly, the ratio of Abeta38/Abeta42 should be an ideal marker not only for a specific diagnosis of AD but also to determine the exact stage of the disease. In addition, it was found that the ratio of Abeta38/Abeta37 from two product lines could enhance the reliability of existing test systems by including a second reading point of the two product lines of gamma-secretase activity.

Based on a FRET approach, the results of the present study moreover provide compelling evidence that APP forms dimers in living cells. Considering that APP homodimers are formed as early as in the ER, the two ectodomain contact sites may initiate the interaction. The two captured polypeptide chains then align and dock in the lipid bilayer where they are further stabilized by consecutive GxxxG motifs of the TMS of APP. This motif provides a template for dimerization of two alpha-helices in a hydrophobic environment. Similar to APP, other gamma-secretase substrates, which have a single GxxxG motif in their TMS were described to form full-length homodimers, such as ErbB-4, E-cadherin, Nectin-1 and APLP-1.

The present work also shows that this interaction is primarily mediated by the glycine residues G29 and G33 of the GxxxG motifs and establishes a crucial functional role of the GxxxG motifs in APP processing. It could be demonstrated that dimeric (wt) as well as mainly monomeric substrates, i.e. beta-CTF mutant G33I, are equally well cleaved by gamma-secretase. For GxxxG mutants an inverse shift in Abeta42 and Abeta38 generation was observed while the Abeta40 level was specifically diminished for the dimerization-disrupting mutant G33I.

Based on a recently presented model, gamma-secretase processing is a continuous process starting from the C-terminal end of the TMS at position 49/48 followed by cleavages at position 46/45 and 42/40 thus removing helix turn by helix turn. Under normal conditions processing of beta-CTF is largely arrested at the step 42/40 leading to high levels of Abeta42 and Abeta40. According to the present data the sequential cleavage model is expanded by proposing that this arrest is due to the dimeric state of the substrate beta-CTF and that the two alpha-helices of the dimeric substrate form a cross-point at residues G29 and G33 leading to a sterical hindrance for gamma-secretase (FIG. 5B). APP TMS dimerization is unique insofar as it is the only known gamma-secretase substrate with a GxxxG motif in triplicate at the start of the TMS. Any variations in APP TMS dimerization strength may fine-tune the actual preferred cleavage site by suspending the sterical hindrance. Candidate factors are non-steroidal anti-inflammatory drugs (NSAIDs) that are reported to affect the generation of different Abeta species like sulindac sulfide and flurbiprofen. Similar to the GxxxG mutants, depending on the NSAID tested they reduce Abeta42 levels and selectively increase Abeta38 production or vice versa. For most NSAIDs the Abeta40 levels also remain unaffected. Thus, some NSAIDs might directly affect the dimerization strength of the substrate beta-CTF. However, any effects of NSAIDs on GxxxG motifs of TMS-7 of PS-1 or TMS-4 or TMS-6 of APH-1 altering the active site conformation might also be possible.

Reduced dimerization strength of beta-CTF allows gamma-secretase to further proceed with cleavages to N-terminal positions generating Abeta38 and Abeta37 (FIG. 5B). Therefore, the present results confirm and expand the recently presented model of the gamma-secretase cleavage model providing a detailed insight into the principal cleavage mechanism of this enzyme (FIG. 5B). In case of APP, dimerization of the TMS through the residues G29 and G33 of the GxxxG motifs determines where the final gamma-secretase cleavage site might occur and if Abeta42 can leave the membrane or shorter forms are produced. This advanced model would predict that gamma-secretase is not only involved in AICD production but has also a function in Abeta-clearance by cleaving off turn by turn until more hydrophilic forms of Abeta are released from the membrane, such as Abeta38.

In summary, according to the experimental observations in the present study two different product lines of gamma-secretase cleavage activities exist, i.e., (I) 49-46-43-40-37-34 and (II) 48-45-42-38-35. These species are generated by independent cleavages of the gamma-secretase starting from the C-terminus and progressing to the N-terminus of the substrate beta-CTF. In AD the levels of products of the second product line are varying whereas the product levels of the first line (I) remain constant. Thus, any ratios between gamma-secretase cleavage products of product line II will provide information on the disease and the stage of the disease. Since levels of the species 42 and 38 are most prominent compared to others of the same line and gamma-secretase is modulated by dimerization of the substrate such that Abeta42 increases in early disease states, it is preferred to take the ratio Abeta38/Abeta42 as a marker.

Finally, it can be concluded that a strong dimerization of the substrate beta-CTF is central to AD since any events stabilizing dimerization would increase the production of Abeta42 that can easily form toxic oligomers. Compounds targeting the dimerization of the APP TMS and interfering with the G₂₉xxxG₃₃ interaction motif of two gamma-secretase substrate molecules may be useful as therapeutics to prevent generation of Abeta42.

### BRIEF DESCRIPTION OF THE DRAWINGS

FIG. 1: APP full-length dimerization is independent of the GxxxG motifs

(A) APP-TMS sequence. Glycine residues of the three consecutive GxxxG motifs are numbered (grey box). The membrane embedded sequence is marked with a yellow box. gamma-secretase cleavage sites are indicated above the sequence by arrows.

(B) Confocal microscopy images of APPwt-YFP and APP-YFP mutants show equal sub cellular localization; bars: 10 μm.

(C) FRET approach: Competition of fluorescent APPwt dimers with APPwt-Flag (means±s.e.m., n=4 with 3-6 cells each). As a negative control, APP-YFP wt does not interact with EGFR-CFP.

(D) FRET efficiencies of wt and mutant APP (means±s.e.m., n=5 evaluated in quadruplicate). Note that APPwt FRET efficiency in C is lower compared to D due to lower amounts of plasmids and thus competition with endogenous APP.

(E) Time course of donor fluorescence recovery (deltaF_{CFP}) during selective photo bleaching of the acceptor (F_{YPP}). Data represent single cells (grey lines) and means (black lines) of a representative cell group.

(F) Linear regression analysis of donor recovery (deltaF_{CFP}) vs. fractional acceptor (YFP) photo bleach. Depicted is the mean of single cells from a representative experiment shown in D.

FIG. 2: The APP TMS dimerizes via the G₂₉XXXG₃₃ motif

(A) Representation of ToxR-system chimeric proteins. The TMS under study is fused between the maltose-binding protein (MBP) and the ToxR transcription activator. Upon dimerization, ToxR-domains initiate transcription of the lacZ gene encoding beta-galactosidase.

(B) ToxR-assay. The tested Abeta residues 29 to 42 wt are indicated. Measured beta-galactosidase activity of the wt-sequence was set as 100% (mean±s.e.m., n=4-6). Mutations to alanine at positions G29 and especially G33 reduce dimerization strength. Mutation G29/33A potentates the effects of the single mutants and G33I acts strongly disruptive. Asterisks indicate significant differences to Abeta29-42 wt (*p <0.01, **p<1×10⁻¹⁵, Student's t-test).

(C) All chimera complement MBP-deficiency of E. coli PD28 cells and allow the cells to grow in minimal media indicating correct membrane integration of chimeric proteins.

FIG. 3: Expression and processing of APP and SPA4CT in stably transfected SHSY5Y cell lines

(A) APP-transfected cells. Antibody 22C11 was used to detect full-length APP (APP). Antibody W0-2 (epitope Abeta residues 2-10) was used to label sAPPalpha (sAPPa) and to immunoprecipitate C-terminal fragments (beta-CTF) and Abeta. Western blot of sAPPbeta was probed with antibody 879 (sAPPb). Vertical bars indicate exchanged lanes for uniform labelling.

(B) SPA4CT-transfected cells. Expression control of immunoprecipitated beta-CTF, Western blot probed with antibody W0-2 (beta-CTF). Western blot of immunoprecipitated Abeta probed with antibody W0-2 (epitope Abeta residues 2-10).

(C) Schematic representation of the processing of APP and the construct SPA4CT. SP: signal peptide (cleaved off in the endoplasmic reticulum); loop: loop region residues 91-111; CBP: Collagen binding site residues 448-465; Abeta: Abeta domain, part of the APP ectodomain and the transmembrane sequence (TM); beta- or alpha-secretases cleave within the Abeta domain to generate soluble APP (sAPPbeta or sAPPalpha, respectively) and C-terminal fragments (beta-CTF or alpha-CTF, respectively). gamma-secretase cleaves beta-CTF within the membrane and generates Abeta.

FIG. 4: TMS dimerization strength determines the gamma-secretase cleavage site and Abeta42 generation

(A, C) Abeta40- and Abeta42-specific ELISAs from media of APP stably transfected SHSY5Y cells and

(B, D) Abeta40- and Abeta42-specific ELISAs from media of SPA4CT stably transfected SHSY5Y cells.

(A-D) The wt was set as 100% (means±s.e.m., n=3-6). Asterisks indicate significant differences to wt (*p<0.01, ** p<0.00001, Student's t-test). Dimerization-attenuating mutants do not affect Abeta40 levels but reduce the Abeta42 level significantly. The mutation G33I reduces Abeta40 as well as Abeta42 levels. The mutants G37A and G38A could not be analyzed by ELISA since the mutations affected the epitope of the monoclonal antibodies used to detect Abeta40 or Abeta42.

(E) MALDI-MS spectra of secreted Abeta from APPwt- or APP G33I-transfected cells. The mutation G33I leads mainly to secreted Abeta37 and Abeta38 at the expense of Abeta40 or Abeta42.

(F) Abeta38-specific ELISA from media of SPA4CT stably transfected SH-SY5Y cells, mutant G29/33A was set as 100% (means±s.e.m., n=4). Asterisks indicate significant differences to wt (*p<0.01, ** p<0.0001, Student's t-test). Abeta38 levels increase when Abeta42 levels decrease of mutants that attenuate dimerization stability.

FIG. 5: Model of the APP TMS and gamma-secretase cleavage mechanism

(A) Computational molecular model of a low-energy conformation of APP TMS with G29 and G33 in the helix-helix interface.

(B) Postulated gamma-secretase cleavage mechanism within the APP TMS. Sequential gamma-secretase cleavage proceeds from the carboxyl terminal end to the N terminus (sites as indicated by scissors). The site of the final cleavage is determined by the strength of interaction of the two helices of two APP molecules mediated by the transmembrane residues G29 and G33. The epsilon- and zeta-cleavages occur independently of the dimeric or monomeric form of the substrate (green scissors). Stable APP-TMS dimers constitute a sterical hindrance for the moving gamma-secretase such that the final cleavage occurs after residue 42/40 and produces Abeta42 and Abeta40 (yellow scissors). Only perturbed dimers or monomeric substrates allow the gamma-secretase to move on to further N-terminal sites yielding Abeta38 and Abeta37 (red scissors).

FIG. 6: Effects of GxxxG mutations are left unimpaired by the APPsw or APParc mutants

GxxxG mutations G33A, G29/33A or G331 were combined together with K670N M671L (sw) or APParc E22G (arc). The levels of sAPPalpha, Abeta42, Abeta40 and Abeta38 were determined in culture medium of stably transfected SH-SY5Y cells. Amounts of sAPPalpha, Abeta42 and Abeta40 were compared to APPwt (set to 100%), Abeta38 was compared to APPsw in D as well as to G29/33A mutant in H and L.

(A-D) APPsw and APParc compared to APPwt.

(E-H) GxxxG mutants in combination with the Swedish mutant.

(I-L) GxxxG mutants combined with the arctic mutant. I: sAPPalpha for arc G331 is n=1. Significance is indicated by the stars in relation to the reference value of 100% (*p<0.001, **p<1*10⁻⁵, n=3-10), bars represent the standard error.

FIG. 7: The APP GxxxG mutant G33A dominates FAD-Presenilin mutations

HEK293 cells were transiently transfected with APP wt or APP G33A in combination with PS-1 wt or mutant forms. ELISAs were used to quantify the levels of Aβ42 and Aβ40.

(A) Aβ40 levels generated by PS-1 wt or mutant forms either from APP wt or APP G33A (APPwt+PS-1 wt was set as 100%, means±s.e.m. n=2-4).

(B) Aβ42 levels generated by PS-1 wt or mutant forms either from APP wt or APP G33A (APPwt+PS-1 wt was set as 100%, means±s.e.m. n=2-4).

FIG. 8: A subset of NSAIDs bind to Aβ

Real time binding studies were done using the BIAcore system. Aβ42 was immobilized on the sensor chip surface and the NSAIDs sulindac sulfide, fenofibrate and ibuprofen were tested for binding.

(A) Sulindac sulfide binds in a concentration dependent manner.

(B) Fenofibrate binds concentration dependently and probably induces a conformational shift.

(C) Ibuprofen does not bind to Aβ as the control solvent DMSO induces the same increase in response units.

The present invention, thus, provides a method for the diagnosis of Alzheimer's Disease (AD) or the stage of AD, said method comprising the following steps:
(a) contacting a sample obtained from a patient with probes specifically binding to at least two gamma-secretase cleavage products Abeta48, Abeta45, Abeta42, Abeta38 and Abeta35; and
(b) determining the amounts of said cleavage products in said sample, wherein an abnormal ratio of said cleavage products compared to a normal ratio is indicative of AD.

The present invention also provides a method for monitoring the progress of an AD therapy, said method comprising the following steps:
(a) contacting a sample obtained from a patient with probes specifically binding to at least two gamma-secretase cleavage products Abeta48, Abeta45, Abeta42, Abeta38 and Abeta35; and
(b) determining the amounts of said cleavage products in said sample, wherein a normalized ratio of said cleavage products as compared to the ratio at the onset of therapy is indicative of a therapeutic effect.

The term “normal ratio” as used herein corresponds to a ratio which can be found in healthy individuals and which is determined by using the average values obtained, e.g., from at least 10, preferably from at least 100 individuals.

Suitable samples for carrying out the diagnostic method of the invention are cerebrospinal fluid (CSF), blood, serum, plasma or urine with CSF and blood being preferred. The sample is taken from the patient using routine methods, e.g. CSF is taken by lumbar puncture.

In preferred embodiments of the above methods, the amounts of Abeta38 and Abeta42 in said sample are determined, wherein a decreased ratio of Abeta38:Abeta42 as compared to the normal ratio is indicative of AD.

In more preferred embodiments of the methods of the invention, the amounts of Abeta38 and Abeta42 in said sample are determined, wherein (i) a decreased ratio of Abeta38:Abeta42 as compared to the normal ratio and an increased concentration of Abeta42 as compared to the normal concentration is indicative of an early stage of AD and (ii) a decreased ratio of Abeta38:Abeta42 as compared to the normal ratio and a decreased or normal concentration of Abeta42 as compared to the normal concentration is indicative of a late stage of AD.

In an even more preferred embodiment of the diagnostic method of the present invention a decreased ratio of Abeta38:Abeta42 means Abeta38:Abeta42≦1.5 preferably ≦1.2 more preferably 1.0.

Any method allowing the determination of the levels of Abeta38, Abeta42 etc. in a sample can be used in the method of the present invention, e.g. immunological assays, size separation, e.g. by gel filtration, amino acid analyses etc. Immunological assays and MALDI-MS are preferred.

In an even more preferred embodiment of the method of the present invention, the determination of the amounts of Abeta38, Abeta42 etc. is carried out by an immunological assay using antibodies specific for Abeta38 etc., i.e., the anti-Abeta38-antibody does not substantially cross-react with Abeta42 and, vice versa, the anti-Abeta42-antibody does not substantially cross-react with hAbeta38. Suitable antibodies are commercially available, e.g. the antibodies G2-10 (anti-Abeta40) and G2-13 (anti-Abeta42) from TGC, Zürich.

The term “antibody” as used herein, preferably, relates to antibodies which consist essentially of pooled monoclonal antibodies with different epitopic specificities, as well as distinct monoclonal antibody preparations. Monoclonal antibodies are made from an antigen containing fragments of the hAbeta42 peptide and hAbeta38, respectively, by methods well known to those skilled in the art (see, e.g., Köhler et al., Nature 256 (1975), 495). As used herein, the term “antibody” (Ab) or “monoclonal antibody” (Mab) is meant to include intact molecules as well as antibody fragments (such as, for example, Fab and F(ab′)₂ fragments) which are capable of specifically binding to protein. Fab and F(ab′)2 fragments lack the Fc fragment of intact antibody.

In a further preferred embodiment of the method of the present invention, the anti-Abeta38-antibody is directed to the C-terminus of Abeta38 and the anti-Abeta42-antibody is directed to the C-terminus of Abeta42 (see also product information on G2-13, TGC, Zurich). Preferably, the antibodies bind to an epitope that is only present on the Abeta species to be detected.

The term “epitope” as used herein is meant to include any determinant responsible for specific interaction with an antibody molecule. Epitopic determinants usually consist of chemically active surface groupings of molecules such as amino acids or sugar side chains and have specific three-dimensional characteristics as well as specific charge characteristics.

The probes, e.g. an antibody, can be detectably labelled, for example, with a radioisotope, a bioluminescent compound, a chemiluminescent compound, a fluorescent compound, a metal chelate, or an enzyme (e.g. horse-radish peroxidase, alkaline phosphatase, beta-galactosidase, malate dehydrogenase, glucose oxidase, urease, catalase etc.) which, in turn, when later exposed to a substrate will react to the substrate in such a manner as to produce a chemical moiety which can be detected. The probes can also be immobilized on an insoluble carrier, e.g. glass, polystyrene, polypropylene, polyethylene, dextran, nylon, natural and modified celluloses, polyacrylamides, agarose and magnetic beads.

Examples of immunoassays suitable for the method of the present invention are competitive or sandwich assays, such as the enzyme linked immunosorbent assay (ELISA), the radioimmunoassay (RIA) or Western Blots. Suitable antibody assay labels are known in the art and include enzyme labels, such as, glucose oxidase, and radioisotopes, such as iodine (¹²⁵I, ¹²¹I), carbon (¹⁴C), sulphur (³⁵S), tritium (³H), indium (¹¹²In) and technetium (⁹⁹mTc), and fluorescent labels, such as fluorescein and rhodamine.

In a further preferred embodiment, the method of the present invention is carried out in an ELISA format with a sandwich-ELISA format being even more preferred. Suitable ELISA and sandwich-ELISA formats are well known to the person skilled in the art.

The method of the present invention can be modified, e.g., by additionally determining the concentration of one or more further biomarkers (e.g., (i) the ratio of Abeta42:Aβ40; (ii) the concentration of Abeta37 or Aβ40; or (iii) the concentration of phosphor-tau (showing an increased concentration in AD patients) which might, in certain cases, further improve the diagnostic value of the method of the invention.

The method of the present invention for monitoring the progress of an AD therapy can be applied to any therapy, e.g., treatment with an NSAID (such as MPC-7869, R-flurbiprofen) or a statin.

For use in the diagnostic research discussed above, kits are also provided by the present invention. Preferably, such kits comprise an anti-Abeta38-antibody and an anti-Abeta42-antibody. The antibodies can be detectably labelled as described above and, preferably, allow diagnosis by ELISA. The kits might contain antibodies which are bound to a solid support, for example, a polystyrene microtiter dish or nitrocellulose paper. Alternatively, said kits are based on a RIA and contain said antibodies marked with a radioactive isotope. In a preferred embodiment of the kit of the invention the antibodies are labelled with enzymes, fluorescent compounds, luminescent compounds, ferromagnetic probes or radioactive compounds.

Finally, the present invention also provides a method of screening for therapeutic agents useful in the prophylaxis or treatment of AD comprising the steps of:
(a) Contacting a test compound with an oligomer, homodimer or monomer of (i) amyloid precursor protein (APP), (ii) beta-CTF, (iii) APP-TMS or (iv) a protein comprising an APP-TMS oligomer, monomer or homodimer; and
(b) determining the formation of oligomers or homodimers from the monomers or the formation of monomers from the homodimers or oligomers,
   wherein a reduced or eliminated formation of oligomers or homodimers from the monomers or the formation of monomers from the oligomers or homodimers indicates that said test compound might be suitable for prophylaxis or treatment of AD.

The test compounds may be very different compounds, both naturally occurring compounds and synthetic, organic and inorganic compounds as well as polymers (e.g. oligopeptides, polypeptides, oligonucleotides and polynucleotides) as well as small molecules, antibodies, sugar, fatty acids, nucleotides and nucleotide analogs, analogs of naturally occurring structures (e.g. peptide “imitators”, nucleic acid analogs, etc.) and numerous other compounds. Test compounds can also be screened for on a large scale, e.g. by screening a very large number being able to contain synthetic or natural molecules. Thus, the test compound of a preferred embodiment of the method according to the invention forms part of a substance library.

A large number of possibly useful test compounds can be screened in extracts of natural products as a starting material. Such extracts may be derived from a large number of sources, e.g. the following species: fungi, actinomycetes, algae, insects, protozoa, plants and bacteria. The extracts showing activity can then be analyzed for isolating the active molecule.

In a preferred embodiment of the screening method of the present invention, the test compound is from a library of fungal extracts. A particularly preferred library of fungal extracts is from a Penicillium or Aspergillus species.

The assay can be carried out in a cell-free extract or in vivo using suitable cell lines. Examples of cells useful for the screening method of the present invention are any bacterial cells containing any reporter gene, e.g., under the control of the ctx ToxR-transcription activator promoter like, e.g., E. coli FHK12. A similar assay could be performed in eukaryotic cells using a fusion construct composed of the TMS and, e.g., an intracellular tyrosine kinase activity. The dimeric state would be reported by the degree of auto-phosphorylation by the tyrosine kinase.

The test compound can be added to the cells via the medium, for example. Fundamentally suited assay formats for identifying positive test compounds are well known in the biotechnological and pharmaceutical industries and additional assays and variations of the above assays provided for the purpose of illustration are obvious to the person skilled in the art.

In a further preferred embodiment of the screening method of the present invention the cells express an oligomer, homodimer or monomer of (i) amyloid precursor protein (APP), (ii) p-CTF, (iii) APP-TMS or (iv) a protein comprising an APP-TMS monomer, homodimer or oligomer in a labelled form in such a way that formation of homodimers or oligomers is not adversely affected, e.g., by use of a reporter gene and allow to monitor formation of oligomers, homodimers or monomers, e.g., by use of the ToxR-system described in the examples, below. The second polypeptide segment of a fusion protein comprising a reporter protein can be a full-length protein or a protein fragment. Proteins commonly used in fusion protein construction include, but are not limited to beta-galactosidase, beta-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions. Further useful assays are binding assays with synthetic peptides, monomers and engineered dimers/oligomers, read-out (fluorescence based) assays allowing high-throughput screening etc.

The screening method according to the invention can be modified by means of protocols described in scientific literature and patent literature. For example, a large number of possibly useful molecules can be screened in a single test. For example, when a field of 1000 compounds shall be screened, in principle all of the 1000 compounds can be placed in a microtitration plate well and tested at the same time. The pool of 1000 can be divided into 10 pools of 100 and the process can be repeated until an individual positive test compound is identified. In any case, the production and simultaneous screening of large libraries from synthetic molecules can be carried out by means of well known methods of combinatorial chemistry.

The following examples illustrate the invention.

### EXAMPLE 1

### General Methods

(A) Plasmids and Transfections

Generation of ToxR-system plasmids has been described (Langosch et al., J Mol Biol 263 (1996), 525-30). APP695 with N-terminal Myc-tag and C-terminal Flag-tag or SPA4CT with C-terminal Flag-tag were used as a template to introduce G25A, G29A, G33A, G29/33A or G33I mutations by site-directed mutagenesis. The cDNAs were inserted into pCEP4 (Invitrogen, Karlsruhe, Germany), which contains a hygromycine-resistance gene. For stable expression, plasmids (2 μg) were transfected into SH-SY5Y cell line (ATCC number: CRL-2266) using Transfectine (Bio-Rad, München, Germany) following the manufacturer's instructions. For FRET-measurements, APP695 cDNAs were amplified with primers allowing in-frame ligation to CFP/YFP in vectors pcDNA3 (Invitrogen). All sequences were confirmed by dideoxy sequencing.

(B) Fluorescence Resonance Energy Transfer (FRET)

For transient expression, plasmids (0.75 μg of pcDNA3-APP695-CFP and 1.5 μg of pcDNA3-APP695-YFP) were transfected into HEK293 cells (ATCC number: CRL1573) using the Fugene 6 transfection reagent (Roche Molecular Biochemicals, Mannheim, Germany) following the manufacturer's instructions. In competition FRET experiments, the amount of plasmid was reduced to 0.35 μg of pcDNA3-APP695-CFP and 0.75 μg of pcDNA3-APP695-YFP to allow addition of up to 1.5 μg of plasmid encoding competitor pCEP4-APP or pcDNA3. FRET measurements were performed as described by Voigt et al., J Biol Chem 280 (2005), 5121-7.

(C) ToxR-System

E. coli FHK12 cells expressing chimera constructs were lysed and beta-galactosidase activity was measured (Langosch et al., 1996). To control correct insertion of fusion proteins into the bacterial inner membrane, chimera were expressed in E. coli PD28 cells deficient for MBP (Brosig and Langosch, Protein Sci 7 (1998), 1052-6). Correct orientated chimera complement MBP deficiency when cells are grown in M9 minimal media with maltose as the only carbon source measured by cell densities after 48 h.

(D) Sandwich ELISA, Immunoprecipitation, and Western Blots

Stably transfected cells were plated at a density of 2×10⁶ cells per 60-mm dish. The day after splitting, 4 ml of fresh media were added and incubated for 24 h. For Abeta40 and Abeta42-specific ELISAs, 50 μl of media were analyzed according to the manufacturer's instruction (The Genetics company (TGC), Basel, Switzerland). The same protocol was applied to determine Abeta38 levels, except that the antibody BAP-29 (provided by M. Brockhaus, Roche, Basel, Switzerland) was used. Abeta was precipitated from equal volumes of conditioned media with anti-serum (generated against residues 1-40). Aliquots of conditioned media were directly analyzed for secreted APP (sAPP). For full-length APP, cells were lysed in a buffer containing 50 mM Tris, pH 7.4, 150 mM NaCl, 2 mM EDTA, 2% NP40 and 2% Triton-X100. For detection of C-terminal fragments (beta-CTF) of APP- and SPA4CT-transfected cells, cells were treated with gamma-secretase inhibitor N-[N-(3,5-difluorophenacetyl-L-alanyl)]-S-phenylglycine t-butyl ester (DAPT) for 24 h, lysed, and equal amounts of protein were immunoprecipitated with an anti-serum (to APP cytosolic domain). Samples were separated by SDS-PAGE, transferred to nitrocellulose and immunolabelled either with antibody W0-2 to Abeta residues 2-10 or 879 (sAPPbeta, provided by P. Paganetti, Novartis, Basel, Switzerland), or antibody 22C11 (Roche, Mannheim, Germany).

(E) MALDI-MS

Abeta from conditioned media was immunoprecipitated with W0-2. Protein-G-Sepharose was washed in the presence of high salt (500 mM NaCl), followed by PBS and 100 mM ammonium chloride, pH 7.4. Abeta was eluted with 50% acetic acid and vacuum-dried. MALDI-MS analysis was carried out on sinapinic acid matrix with an UltraflexII TOF/TOF (Bruker Daltonics, Bremen, Germany.)

(F) Molecular Modelling

For both helices five different, non-symmetrical starting conformations with identical backbone but varying random side chain conformations were generated. From each starting conformation, 65,000 structures were generated by randomly rotating the helix 0-360° around the long axis; translating it −2 to +6 Å toward or away from geometric centre, respectively, translating each helix ±10 Å along its long axis and tilting the helices relative to the diad axis by ±20°. A dielectric constant of 20 was used for the electrostatics and an energy map was created. The rotation was sampled in 36 degree bins, the tilt in 2.5 degree bins, the longitudinal shift in 2 Å bins and the translational shift was sampled in 0.8 Å bins. The average energy of each bin was evaluated independently by using Boltzmann averaging. The energy map was graphically evaluated, and a symmetric structure was assumed, so that energy minima corresponding to asymmetric structures were disregarded. A related procedure has been shown to reliably model helix bundles (Gottschalk, J Mol Graph Model 23 (2004), 99-110).

(G) Biacore

Abeta peptides were dissolved to 50 μg/ml in 10 mM NaOAC, pH 3.4 and used for covalent coupling to the sensor chip surface CM5 (BIAcore amine coupling kit). This resulted in 2000 response units (RU) provided by immobilized Abeta peptides. The NSAIDs were dissolved in 100% DMSO and diluted in 1×PBS/0.005% P20. The injection of NSAIDs in a volume of 10 μl was followed by a dissociation phase of 600 sec and the injection of 5 μl 20 mM HCl to regenerate the surface. Baseline stability was tested by the injection of 210 μl of running buffer (1×PBS/0.005% P20). For all experiments a flow rate of 30 μl/min was chosen according to the manufacturers' instructions.

### EXAMPLE 2

### Impact of the GxxxG Motifs an Full-Length App Homodimerization

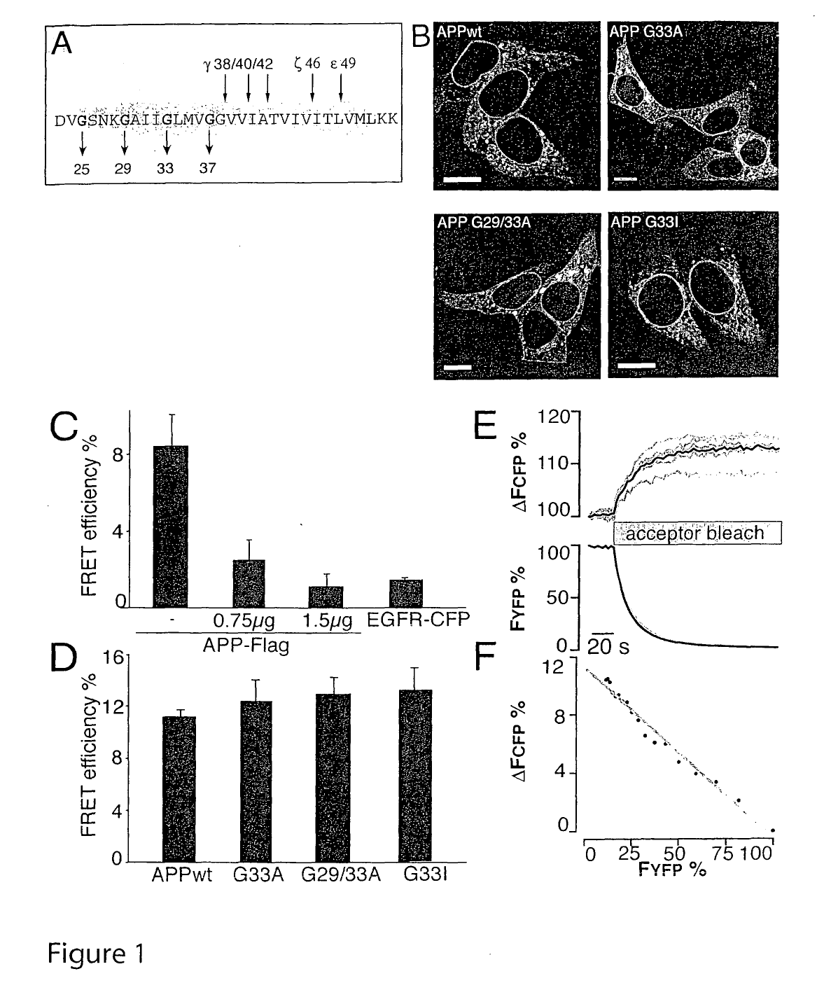

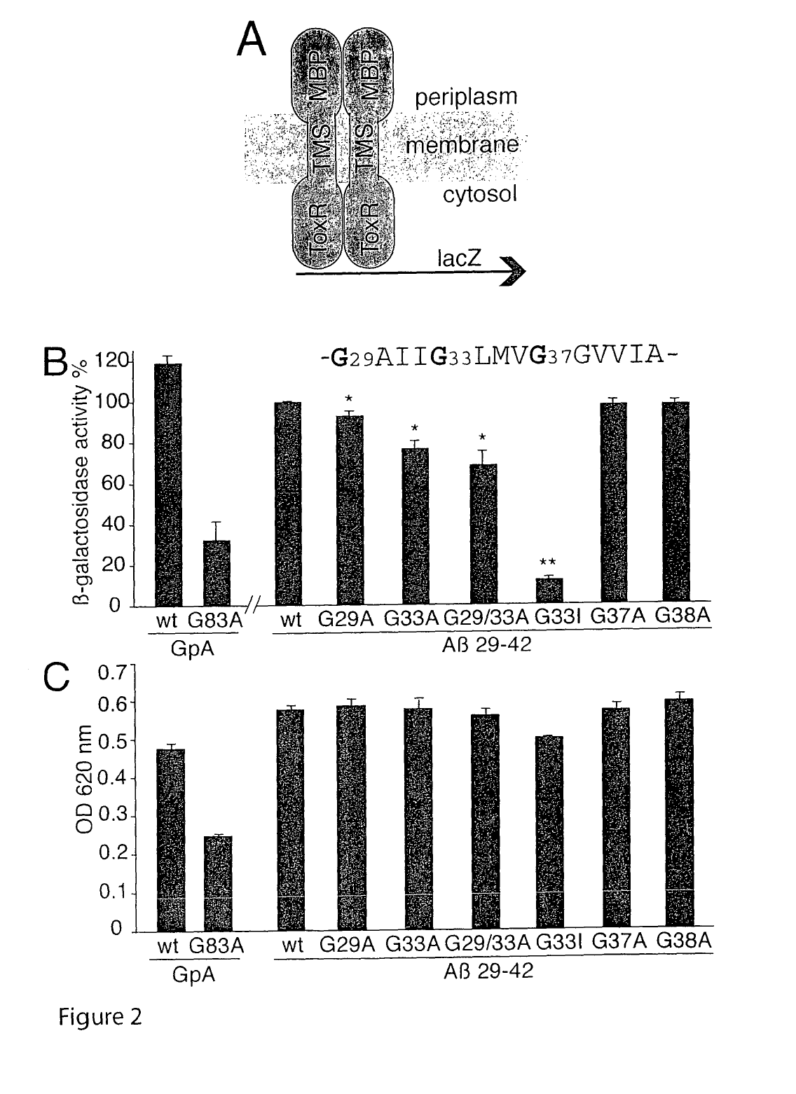

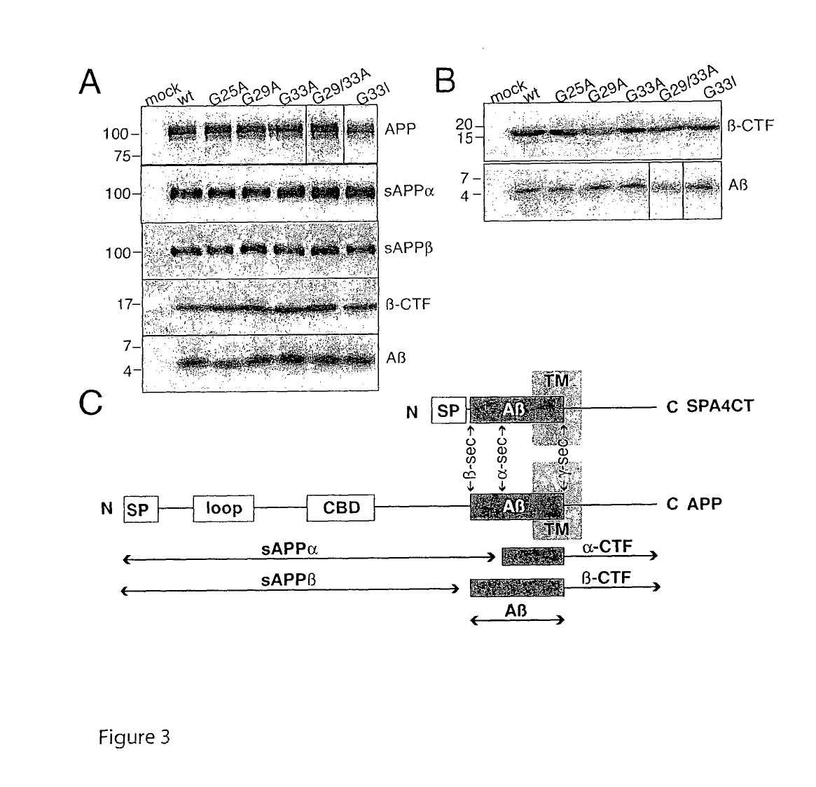

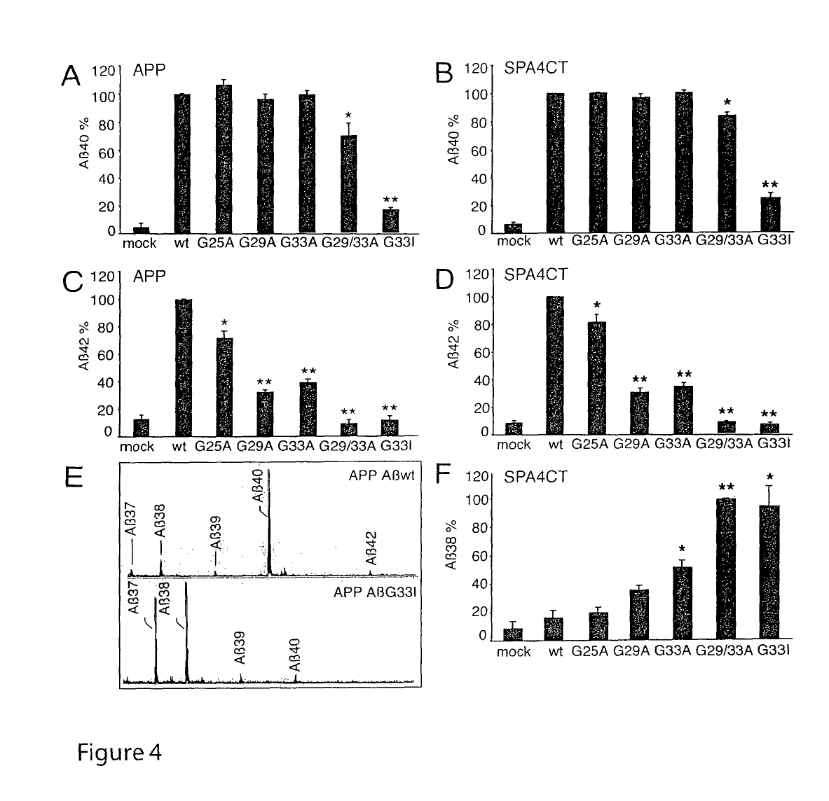

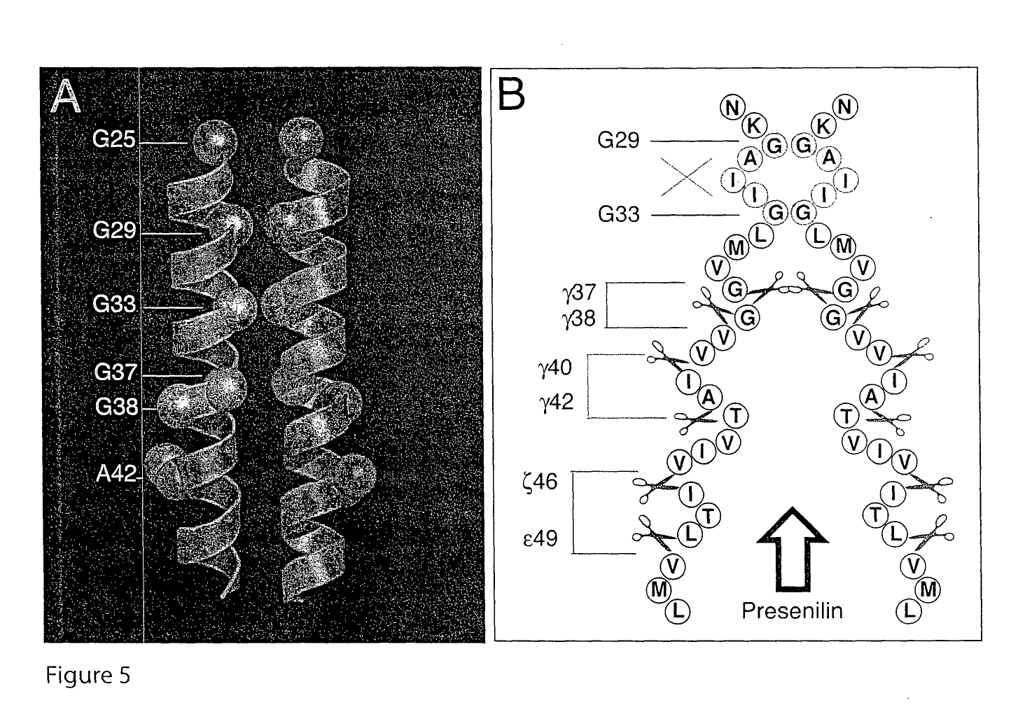

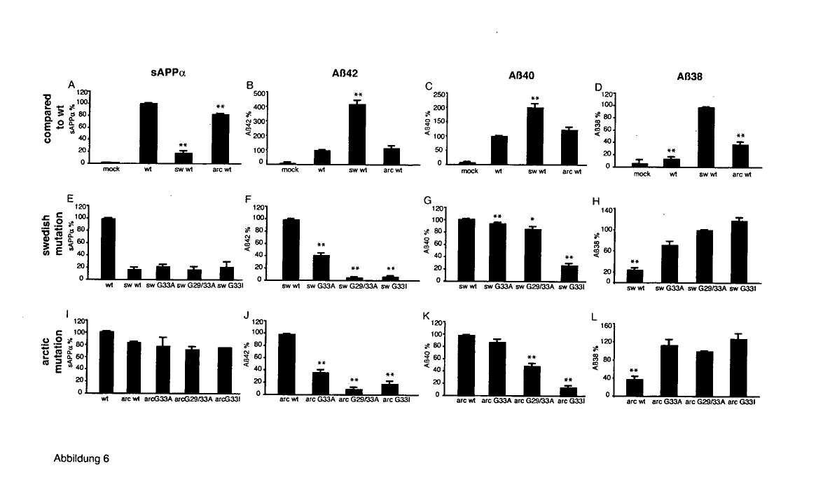

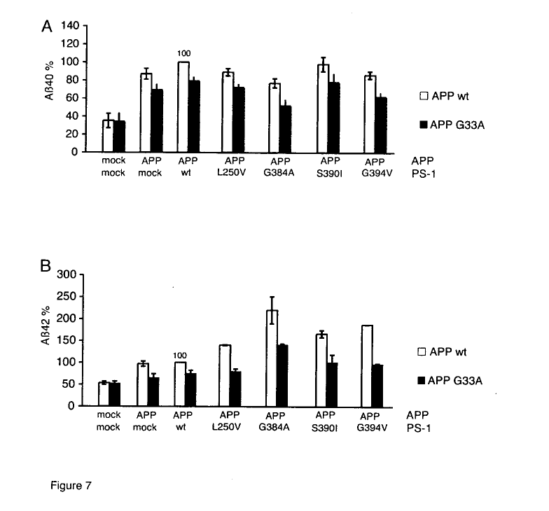

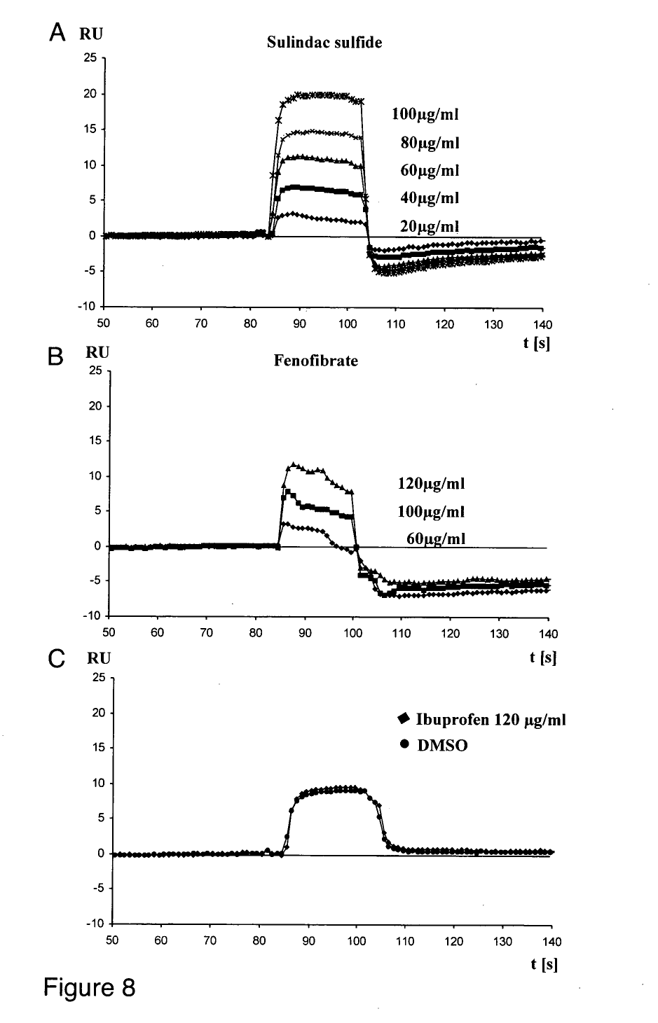

Biochemical studies had previously indicated the APP ectodomain in homodimerization by two different sites, one located at amino acids 448-465 that is overlapping with the collagen binding site and another at the loop region encompassing residues 91-111 (Beher et al., J Biol Chem 271 (1996), 1613-20; Scheuermann et al., J Biol Chem 276 (2001), 33923-9). The GxxxG motifs would therefore provide a third contact site of APP dimers. A fluorescence resonance energy transfer (FRET) approach was used to examine whether APP dimerization also depends on the GxxxG motif in living cells. Cyan or yellow fluorescent proteins (CFP or YFP) were fused to the C terminus of full-length APP695 wild-type (wt) or mutant constructs G33A, G331 or G29/33A, which are mutated within the central GxxxG motif. All fusion proteins showed identical subcellular distribution patterns in HEK293 cells as assessed by confocal microscopy (FIG. 1B). For APPwt-YFP and APPwt-CFP, a FRET efficiency of 11.2% was determined, demonstrating for the first time close proximity of two APP molecules in living cells (FIG. 1D). Since FRET signals were concentration-dependently reduced upon co-expression of non-fluorescent APPwt-Flag, it was concluded that the FRET signals observed are specific (FIG. 1C). The specificity is further indicated by APP-YFP and the co-localization of the unrelated EGFR-CFP, a family member of the gamma-secretase substrate ErbB-4, that does not show any detectable FRET efficiency, as expected (FIG. 10). The kinetics of FRET and regression analysis of acceptor photobleaching are shown in FIGS. 1E and 1F. To test the influence of the APP TMS GxxxG motifs an APP homodimerization, FRET efficiencies of APP G33A, APP G29/33A and APP G33I were measured. No major differences were observed for the mutants compared to the wt (FIG. 1D). It is concluded that mutations of the GxxxG motif do not influence APP dimerization in general. Accordingly, GxxxG-mediated interactions seem to be secondary to the initiation of dimerization through the N-terminal contact sites (Beher et al., (1996); Scheuermann et al., (2001)) and might rather affect the TMS conformation within the membrane.

### EXAMPLE 3

### The App Transmembrane Sequence Dimerizes Via the GxxxG Motifs

To selectively analyze the ability of the APP GxxxG motifs as part of the TMS to promote helix self-interaction, the ToxR-system (Langosh et al., (1996)) was applied, an assay to measure the homodimerization strength of a short TMS within the bacterial inner membrane. To this end, a fusion protein was constructed composed of the periplasmic maltose-binding protein (MBP), the TMS under study, which integrates into the bacterial inner membrane, and the cytosolic transcription activation domain ToxR (FIG. 2A). TMS-driven dimerization within the membrane leads to close proximity of the ToxR-domains, which are able to activate transcription of the reporter gene encoding beta-galactosidase only in the dimeric state. The dimerization strength of the TMS under study is thus reported by beta-galactosidase activity. TMS Abeta residues 29 to 42 gave a strong signal similar to that obtained with residues 75 to 87 of glycophorin A (GpA) (FIG. 2B), a well-investigated self-interacting TMS (Langosch et al., (1996)). Dimerization strength was gradually reduced from Abeta TMS mutant constructs G29A and G33A to mutation G29/33A and was abolished by the mutation G33I (FIG. 2B). Unfortunately, the influence of the juxtamembrane residue G25 cannot be measured by this system as only TMS sequences can be analyzed. Since the mutations G37A and G38A distal to the central GxxxG motif did not cause any effects, it is concluded that G29 and G33 are the key residues of the GxxxG motifs that determine the association of Abeta TMS. The differences between the double mutation G29/33A and mutation G33I might directly reflect the varying affinities of the two helices. While the mutation G29/33A might still allow the two TMS to approximate each other, stabilizing van der Waals bonding might occur. In contrast, the branched amino acid isoleucine of the G33I mutant might prevent this approximation and thus abolish dimerization. All ToxR-chimera inserted correctly into the bacterial membrane (FIG. 2C) and showed equal expression levels (data not shown).

### EXAMPLE 4

### GxxxG Mutations Reduce Abeta42 Levels

Next it was examined whether the GxxxG mutations alter APP processing and generation of Abeta. SH-SY5Y cells stably expressing APP695 wt or mutants G25A, G29A, G33A, G29/33A, or G33I were generated. For all cell lines, similar levels of full-length APP, soluble APP (sAPPalpha and sAPPbeta), beta-CTF and total secreted Abeta were observed by Western blot (FIG. 3, A and C). Thus, GxxxG mutations neither affect shedding by beta- or alpha-secretases nor interfere with maturation and surface expression of APP. However, when Abeta42, Abeta40 and Abeta38 species were quantified in a Sandwich enzyme linked immunosorbent assay (ELISA), significant differences to the wt were revealed. The mutants G25A, G29A and G33A showed no alterations in Abeta40 levels, but Abeta42 secretion was significantly reduced by 28%, 67%, and 60%, respectively, compared to cell lines expressing APPwt (FIG. 4, A and C). Thus, G29A and G33A reduced Abeta42 levels to a similar extent, although G33A attenuated dimerization of the TMS more efficiently than G29A (FIG. 2B). This might be due to the bacterial membrane insertion of Aβ residues 29 to 42 with G29 located at the boundary of the chimera constructs used in the ToxR-assay (FIG. 2B). In addition, residue G25 at the extracellular juxtamembrane position appears to be an extended part of the GxxxG dimerization motif since the G25A mutant reduced Abeta42 levels similar to mutations G29A and G33A. The double mutation G29/33A slightly reduced Abeta40 generation and decreased the Abeta42 level to the background secretion of mock-transfected cells (FIG. 4, A and C). Since the mutation G33I abolished the generation of Abeta40 and Abeta42 but no reduction of total Abeta was observed by Western blot (FIG. 3A) it was assumed that this mutant might lead to the secretion of other Abeta species (see Example 6).

### EXAMPLE 5

### Effects of GxxxG Mutants are Independent of the APP Ectodomain

To exclude influences of the APP ectodomain contact sites or beta-secretase activity an Abeta generation of GxxxG mutants, secreted Abeta of SH-SY5Y cells stably transfected with the construct SPA4CT (Dyrks et al., FEBS Lett 309 (1992), 20-24) were analyzed, which releases beta-CTF after cleavage of the signal peptide (SP) independently from gamma-secretase (FIG. 3C). SPA4CTwt- or mutant transfected cells showed equal expression levels of beta-CTF and Abeta by Western blot analysis (FIG. 3B). Generation of Abeta40 and Abeta42 of SPA4CTwt and mutants yielded similar levels of Abeta as gained from the corresponding full-length APP constructs, described above (FIG. 4, B and D). This indicates that the effect of the GxxxG motif an Abeta production is independent from the APP ectodomain.

### EXAMPLE 6

### Abeta38 is Produced at the Expense of Abeta42

Since total Abeta levels from APPwt and G331 and G29/33A mutants appeared similar as observed by Western blot (FIG. 3A), samples were analyzed for the presence of C-terminally truncated Abeta species by MALDI-MS. In the culture supernatant from APP G33I-expressing cells Abeta38 and Abeta gave the most prominent signals whereas Abeta40 could hardly be detected (FIG. 4E). Abeta from APP G29/33A-expressing cells also showed a much stronger signal for Abeta38 (data not shown). To investigate if the seemingly increasing amounts of Abeta38 (FIG. 4E) are produced at the expense of Abeta42, the level of Abeta38 secreted from beta-CTF-expressing cells was determined by using an Abeta38 specific ELISA (FIG. 4F). The mutations G29/33A and G33I showed the highest amount of Abeta38 produced (mutation G29/33A was set as 100%) corresponding to their abolished Abeta42 generation and reduced Abeta40 levels. Compared to the double mutation G29/33A the single substitutions G29A and G33A generated less Abeta38 (36% and 52%, respectively) as they specifically reduced Abeta42 generation but not Abeta40. The mutation G25A showed no significant change in Abeta38 levels compared to the wt. The mutant G33I led to slightly less Abeta38 produced compared to mutant G29/33A but instead led to increased amounts of Abeta37 (FIG. 4E). Thus, there is a strong inverse correlation between the generation of Abeta42 and Abeta38 depending on the GxxxG mutations. The gradually decreasing or increasing levels of Abeta42 and Abeta38 clearly depend on the individual glycine substitutions and are either attributed to conservative or non-conservative mutations in their consequence. The data indicates that the Abeta42 and A38 production is intimately linked to the dimerization strength of the TMS-TMS interaction through the residues G29 and G33 of the GxxxG motifs as indicated by the ToxR-assay. The results show that mutations of the GxxxG motifs do not affect cleavage efficiency but shift the gamma-secretase cleavage to more N-terminal sites.

### EXAMPLE 7

### A Helix-Helix Interface with G29 and G33 is Favoured Conformationally

In order to test whether a helix-helix interaction through the GxxxG motif is favoured energetically and conformationally, a computational search of conformations of Abeta residues 25 to was performed. A low-energy conformation with a right-handed crossing angle and all four glycines of the three consecutive GxxxG motifs at the helix-helix interface was observed (FIG. 5A). According to this model, residues G29 and G33 constitute the core of the interface with Calpha-Calpha distances of less than 5 Å for each pair compared to the distances of G25 (6.7 Å) and G37 (5.7 Å).

### EXAMPLE 8

### The GxxxG Motif has an Impact on Known Mutations Around Beta- and Alpha-Secretase Sites Outside and in the APP TMS that Cause AD and Usually Lead to an Enhanced Abeta42 Production

In this example it is demonstrated that the GxxxG motif has an impact on known mutations around alpha- and beta-secretase sites outside and in the APP TMS that cause AD and usually lead to an enhanced Abeta42 production. The so called Swedish mutant K670N M671L (APPsw) located N-terminally to the beta-secretase cut {Mullan et al., 1992, Nat Genet, 2, 340-34} and the arctic mutant E22G (APParc) C-terminally to the alpha cut were analyzed {Kamino et al., 1992, Am J Hum Genet, 51, 998-1014; Nilsberth et al., 2001, Nat Neurosci, 4, 887-893}. The Swedish mutant K670N M671L renders APP into a better substrate for BACE which leads to a 3- to 6-fold increased production of Abeta {Citron, 1992, Nature, 360, 672-674; Citron, 1994, Proc Natl Acad Sci USA, 91, 11993-11997}.

The arctic mutation E22G together with the dutch mutation E22Q and the Italian mutation E22K, the flemish A21G and the Iowa-mutation E23N are all centered in the middle of the Abeta-sequence, C-terminal to the alpha-cleavage site (at position 16). Patients with the arctic mutation E22G, have lower concentrations of soluble Abeta42 and Abeta40 in blood and secreted Abeta42 in cell culture supernatant from APPE22G transfected cells is decreased by 19% {Nilsberth et al., 2001, Nat Neurosci, 4, 887-893}. Since BACE processing of APP E22G is increased, alpha-secretase activity is decreased {Stenh et al., 2002, Neuroreport, 13, 1857-1860}. The effect on neuronal cell death is enhanced by the physicochemical properties leading to an accelerated formation of protofibrils {Nilsberth et al., 2001, Nat Neurosci, 4, 887-893}.

First, APP processing of APPwt and the Swedish or arctic mutant APP was analyzed in transfected SH-SY5Y cells (FIG. 6 A-D). ELISA was used to determine levels of sAPPa, Abeta42, Abeta40 and Abeta38. Compared to APPwt in APPsw transfected cells soluble APP (sAPPa) was found reduced to 18% and for APParc to 83%. This indicates that APPsw and APParc forms are shifted into the beta-secretory pathway (FIG. 6 A). In contrast to published results a slight increase of soluble Abeta42 (by 15%) and Abeta40 (by 22%) was found (FIGS. 6 B, C) {Nilsberth et al., 2001, Nat Neurosci, 4, 887-893; Stenh et al., 2002, Neuroreport, 13, 1857-1860}. This might be due to differences in the cell lines used (SH-SY5Y instead of HEK293). Also APPsw leads to an increase of total amounts of Abeta as observed by others but we found also a change in the ratio of Abeta40/Abeta42 produced. Whereas the amount of Abeta40 increased by a factor of 2 (FIG. 6 C), the increase of Abeta42 was 4-fold (FIG. 6 B). The effect on the ratio in favor of Abeta42 is a novel finding and indicates a specific role of BACE in the production of the aggregation-prone Abeta42. In addition, the amount of Abeta38 produced was quantified. Since the Aβ38 levels produced from APPwt transfected cells were close to the detection limit the level measured in APPsw supernatants was set to 100% for easier comparison (FIG. 6 D). Compared to APPwt Abeta38 was increased by 85% in APPsw and found 2-fold increased in APParc transfected cells.

When GxxxG mutations were combined with APPsw or APParc sAPPa levels remained unimpaired and resembled those of the FAD mutants (APPwt was set to 100%) (FIG. 6 E, I). Thus, GxxxG mutants neither had an influence on BACE nor on alpha-secretase activity.

To quantify the effects of FAD-GxxxG mutations, Abeta42 and Abeta40 levels secreted from APPsw and APParc constructs and Abeta38 levels from APPsw G29/33A or APParc G29/33A were set to 100%. Abeta levels measured from FAD-GxxxG mutants were similar to levels of GxxxG mutants alone (FIG. 6 F-H, J-L). APPsw G33A and APParc G33A showed 60% less Abeta42. Abeta42 from GxxxG mutants G29/33A and G33I were below the detection limit. Abeta40 levels from APPsw G33A and APParc G33A mutant constructs were similar to APPsw or APParc, although these were slightly decreased to 93% and 89%, respectively (FIG. 6 G, K). The double mutant FAD-G29/33A generated significantly less Abeta40 (APPsw G29/33A 84% and APParc G29/33A 50% of APPsw or APParc alone). Nevertheless, this resembled the amounts of Abeta40 produced by the GxxxG mutant G29/33A, which releases 70% of Abeta40 compared to APPwt.

The level of Abeta40 released from FAD-APP G33I transfected cells is similar to mock-transfected cells. Abeta38 levels are increased in APPsw or APParc transfected cells to the same extent as measured for GxxxG mutants alone (FIG. 6 H, L).

Taken together, (i) GxxxG mutants and (ii) GxxxG mutants in combination with familial AD(FAD)-mutants yielded similar levels of soluble Abeta. This indicates that GxxxG effects are superior to FAD-mutants in the amyloid cascade and thus underline the importance of the GxxxG mediated dimerization for the generation of amyloid and for any therapeutic efforts on amyloid inhibition.

Whereas the Swedish and the arctic mutations have an influence on the initial processing of APP by beta- or alpha-secretases, resulting products remain unimpaired by the GxxxG motif. This motif specifically has an effect on the gamma-secretase activity downstream in the processing cascade and lowers the production of Abeta42, Abeta40 while increasing Abeta38 and shorter forms.

### EXAMPLE 9

### PS-1 Familial AD Mutations and GxxxG

GxxxG mutations are superior to APP FAD mutations as demonstrated for APP sw and APP arc mutations. In addition to APP FAD mutations also occur in presenilin 1 (PS1) leading to early onset phenotypes of the disease. To examine weather the APP GxxxG motif can rescue the Abeta42 enhancing effects of PS1 FAD mutations we transiently co-expressed APP wt or APP G33A together with PS-1 wt or FAD mutants in HEK 293 cells and analyzed the Abeta production. The following PS-1 FAD mutations were generated: L250V in TMS6 and G384A, 53901 and G394V in TMS7 {Campion et al., 1999, Am J Hum Genet, 65, 664-670; Cruts et al., 1995, Hum Mol Genet, 4, 2363-2371; Furuya et al., 2003, J Neurol Sci, 209, 75-77; Rogaeva et al., 2001, Neurology, 57, 621-625}.

The Aβ40 levels generated from PS-1 wt co-expressed with either APP wt (set as 100%) or APP G33A differ between measurements by about 20% (FIG. 7A). This difference might be due to the cellular system (HEK293 instead of SH-SY5Y cells) or due to the method of transient transfection. Analyses revealed that Aβ40 levels remained uninfluenced, independent of the form of APP overexpressed, APP wt or APP G33A and independent of the PS-1 mutations (FIG. 7A).

In contrast, the Aβ42 levels of APP wt were remarkably found increased for the PS-1 mutants, as expected (FIG. 7B). APP G33A decreased the level of Aβ42 below the level that is observed when PS-1 wt is co-expressed (FIG. 7B). The enhancing effect of PS1 mutants on Aβ42 levels could be even more than rescued, most likely due by affecting directly the substrate/enzyme interaction of APP/PS1. This illustrates the feasibility of the present approach to target not only the homodimerization of the gamma-secretase substrate APP to inhibit Aβ production but also to target APP/PS1 interaction through the GxxxG motif.

### EXAMPLE 10

### GxxxG and NSAIDs

As shown above, certain mutations of the GxxxG motifs within the APP TMS, like G25A, G29A and G33A lead to a significant decreased production of Abeta42 and an increased level of Abeta38 released by living cells without significantly alterations of Abeta40 levels. This effect on Abeta production is also observed for APP transfected cells treated with non-steroidal anti-inflammatory drugs (NSAIDs). For example, the specific NSAID sulindac sulfide reduces Aβ42 generation, increases Aβ38 levels without affecting Aβ40 levels {Weggen et al., 2001, Nature, 414, 212-216}. Likewise, Ibuprofen lowers Aβ42 levels and fails to affect Aβ40 production. Unfortunately, Aβ8 levels were not analyzed {Gasparini et al., 2004, J Neurochem, 91, 521-536; Weggen et al., 2001, Nature, 414, 212-216}. Both findings were made independently and point to a similar allosteric modulation of the enzyme complex {Beher et al., 2004, J Biol Chem, 279, 43419-43426; Takahashi et al., 2003, J Biol Chem, 278, 18664-18670}. Interestingly, for another subset of NSAIDs, the opposite mode of action was described as for example for Fenofibrate {Kukar et al., 2005, Nat Med, 11, 545-550}. Fenofibrate drastically increased Aβ42 levels, decreases Aβ38 generation but also had no effect on Aβ40 levels.

According to above characterization of the GxxxG function as a helix-helix interaction motif (Munter et al., 2007, Embo J, 26, 1702-1712) we postulated a potential binding of the Abeta modulating NSAIDs to the Aβ region encompassing this motif. Similarly to the GxxxG mutants we hypothesize either a dimer-stabilizing or an attenuating effect of gamma-secretaes substrates. Both effects would require a direct binding of NSAIDs to the Abeta region if helix-helix interactions would be involved. Thus, a real time binding approach using the BIACORE system was performed (FIG. 8). The NSAIDs sulindac sulfide and Fenofibrate were dissolved in DMSO and diluted in PBS with detergent (1×PBS/0.005% P20). As hypothesized, both NSAIDs bound to immobilized Aβ in a dose-dependend manner compared to control solvents (FIG. 8A, B). For Fenofibrate a conformational change upon binding could be observed as an additional effect (FIG. 8B). The injections of Ibuprofen did not bind reveal a specific binding to Aβ, which could be due to an affinity that is below the detection limit (FIG. 8C).

Nevertheless, this indicates specific binding of NSAIDs to the Aβ region even in the absence of a membrane environment. It is tempting to assume that while sulindac sulfide might attenuate the dimer stability of Abeta, fenofibrate might stabilize dimers.

What is claimed is:

1. A method for the diagnosis of Alzheimer's Disease (AD) or the stage of AD, said method comprising the following steps:
(a) contacting a sample obtained from a patient with probes specifically binding to at least two gamma-secretase cleavage products Abeta48, Abeta45, Abeta42, Abeta38 and Abeta35; and
(b) determining the amounts of said cleavage products in said sample, wherein an abnormal ratio of said cleavage products compared to a normal ratio is indicative of AD.

2. A method for monitoring the progress of an AD therapy, said method comprising the following steps:
(a) contacting a sample obtained from a patient with probes specifically binding to at least two gamma-secretase cleavage products Abeta48, Abeta45, Abeta42, Abeta38 and Abeta35; and
(b) determining the amounts of said cleavage products in said sample, wherein a normalized ratio of said cleavage products as compared to the ratio at the onset of therapy is indicative of a therapeutic effect.

3. A diagnostic method for distinguishing between Alzheimer's Disease (AD) and non-AD, said method comprising the following steps:
(a) contacting a sample obtained from a patient with probes specifically binding to the gamma-secretase cleavage products Abeta42, Abeta40 and Abeta38 and (ii) to; and
(b) determining the amounts of said cleavage products in said sample,
wherein a ratio of Abeta38:Abeta42 >1 in combination with a normal ratio of Abeta42:Abeta40 is indicative of non-AD.

4. The method of claim 1 or 2, wherein the ratio of Abeta38:Abeta42 is determined.

5. The method of claim 1, wherein (i) a decreased ratio of Abeta38: beta42 as compared to the normal ratio and an increased concentration of Abeta42 as compared to the normal concentration is indicative of an early stage of AD and (ii) a decreased ratio of Abeta38:Abeta42 as compared to the normal ratio and a decreased or normal concentration of Abeta42 as compared to the normal concentration is indicative of a late stage of AD.

6. The method of claim 4 or 5, wherein the ratio Abeta38:Abeta42 is ≦1.5.

7. The method of any one of claims 1 to 6, wherein the sample is CSF or blood.

8. The method of any one of claims 1 to 6, wherein the probes are antibodies.

9. The method of claim 8, wherein the antibodies are monoclonal antibodies.

10. The method of claim 9, wherein the method is carried out as ELISA.

11. The method of claim 10, wherein the ELISA is a sandwich-ELISA.

12. The method of any one of claims 1 to 11, comprising the determination of the concentration of at least one additional biomarker.

13. The method of claim 2, wherein therapy comprises treatment with an NSAID or statin.

14. The method of claim 13, wherein the NSAID is MPC-7869 (R-flurbiprofen).

15. A kit suitable for carrying out the method according to any one of claims 1 to 14 comprising at least two anti-Abeta-antibodies.

16. The kit of claim 15 comprising at least an anti-Abeta38-antibody and an anti-Abeta42-antibody.

17. A method of screening for a therapeutic agent useful in the prophylaxis or treatment of AD comprising the steps of
(a) contacting a test compound with an oligomer, homodimer or monomer of (i) amyloid precursor protein (APP), (ii) beta-CTF, (iii) APP-TMS or (iv) a protein comprising an APP-TMS monomer, homodimer or oligomer; and
(b) determining the formation of oligomers or homodimers from the monomers or the formation of monomers from the homodimers or oligomers,
wherein a reduced or eliminated formation of oligomers or homodimers from the monomers or the formation of monomers from the oligomers or homodimers indicates that said test compound might be suitable for prophylaxis or treatment of AD.
